(19) United States
(12) Patent Application Publication
(10) Pub. No.: US 2016/0296628 A1
(43) Pub. Date: Oct. 13, 2016

(54) NANOPARTICLE DELIVERY SYSTEMS FOR CYTOLYTIC PEPTIDE PRODRUGS

(71) Applicant: Washington University, St. Louis, MO (US)

(72) Inventors: Samuel A. WICKLINE, St. Louis, MO (US); Andrew JALLOUK, St. Louis, MO (US); Paul SCHLESINGER, University City, MO (US); Hua PAN, St. Louis, MO (US)

(73) Assignee: Washington University, St. Louis, MO (US)

(21) Appl. No.: 15/004,767

(22) Filed: Jan. 22, 2016

Related U.S. Application Data
(60) Provisional application No. 62/107,243, filed on Jan. 23, 2015.

(51) Int. Cl. A61K 47/42; A61K 9/14; C12Q 1/37; A61K 31/02

(52) U.S. Cl. CPC A61K 47/42; A61K 31/02; A61K 9/146; C12Q 1/37

(57) ABSTRACT

Compositions and methods of treatment using conjugates of pore-forming compounds coupled to blocking segments by protease-cleavable linkers and absorbed onto lipid-based delivery vehicles are described. These compositions and methods permit personalization of treatment.

### RELATED APPLICATION

This application claims priority from U.S. 62/107,243 filed 23 Jan. 2015, the contents of which are incorporated herein by reference in its entirety.

### ACKNOWLEDGEMENT OF GOVERNMENT SUPPORT

The subject matter herein was supported by a grant from the National Institutes of Health NIH, R01 HL073646. The U.S. government has certain rights in this invention.

### TECHNICAL FIELD

The invention relates to delivery of prodrugs of pore-forming, cytolytic compounds, typically peptides. More specifically, the invention relates to delivery of such prodrugs using self-assembling emulsions of liposomes or of other particles such as nanoparticles wherein a hydrophobic core is coated with a lipid/surfactant layer.

The design of the prodrugs and their delivery on particulate vehicles permits individualized, personalized treatment of subjects harboring tumors or other cancers.

### BACKGROUND ART

U.S. Pat. Nos. 7,943,168 and 8,496,945 incorporated herein by reference, describe delivery of pore-forming, cytolytic peptides using optionally targeted “oil-in-water” emulsions, typically emulsions of nanoparticulate fluorocarbon cores coated with lipid/surfactant layers, wherein the lipid/surfactant layer contains the peptide to be delivered. The nanoparticles provide a delivery system that is helpful in overcoming lack of specificity of the activity of the peptides, but improvements are still needed in order to enhance this specificity. In the present application, such peptides are supplied as prodrugs where they can be activated by proteases unique to target organs or tissues for which the peptides are intended, and wherein the delayed release of a cytolytic compound (which is usually a peptide) permits a wider range of particulate carriers. Thus, it is only upon activation of the cytolytic compound by virtue of the action of a protease that the pore-forming/cytolytic activity of the compound is realized. As the pore-forming compounds are supplied as prodrugs, in the present invention, in addition to nanoparticles, liposomes may also be used as delivery vehicles. The literature describing liposomes and other particulate formations is well known. Liposomes are commonly used as delivery vehicles for drugs of various types.

It is known to prepare prodrugs of certain cytolytic/pore-forming peptides. For example, LeBeau, A. M., et al., Mol. Cancer Ther. (2009) 8:1378-1386 describe prodrugs of melittin, a 26 amino acid amphipathic cytolytic peptide from bee venom wherein the cytolytic peptide is coupled to a propeptide sequence through a domain that is a substrate for fibroblast activation protein α (FAP). According to the article, FAP is a membrane-bound serine protease expressed on the surface of fibroblasts present within the majority of human epithelial tumors but not expressed by normal tissue. The authors showed that these prodrugs had antitumor activity. Efforts were made to optimize the prodomain amino acid sequence based on the prodomain of melittin itself. As noted, the linker to melittin included a sequence cleavable by FAP.

In addition, Holle, L., et al., Int. J. Oncol. (2009) 35:829-835 describe a conjugate in which the latency associated peptide (LAP) derived from TGF-β was fused through a cleavage site for matrix metalloproteinases (MMP's) to melittin.

Neither of these publications describe systems that overcome stability and biodistribution problems associated with these prodrugs. The present invention solves these problems by delivering similar conjugates using particulate delivery vehicles.

The present inventors have presented a poster describing one embodiment of a composition of the invention at the Experimental Biology 2014 conference in April of 2014, along with an abstract describing this work.

### DISCLOSURE OF THE INVENTION

The problems associated with administering prodrug conjugates of cytolytic peptides have been solved by the present invention by associating the prodrugs with particulate delivery vehicles. In one embodiment, the prodrugs are associated with lipid/surfactant layer surrounding a hydrophobic core of a nanoparticle, and optionally targeted to a tissue, for example one whose demise is desired. In another embodiment, the delivery vehicles may be micelles, liposomes or other lipid-based microparticles or nanoparticles, which also can optionally comprise a targeting agent. Delivery to a target cell or tissue can be effected so that the pore-forming/cytolytic peptide can inhibit the growth of the target cells or effect cell death while the surrounding tissues and non-target cells are unaffected. This is the case for several reasons.

First, the association with the nanoparticle or other lipid-based particle protects the prodrug from degradation. Second, a multiplicity of prodrugs can be associated with a single particle, thus resulting in sustained delivery. Third, by association with a particulate delivery system, the rate of drug release can be controlled by means not otherwise available as will be further described below. The pore-forming/cytolytic compound, such as a peptide, can also facilitate intracellular delivery of other therapeutic or diagnostic agents.

The invention is thus directed to compositions and methods for delivering prodrug conjugates of cytolytic compounds by means of self-assembling nanoemulsions of lipid-based delivery vehicles. In some embodiments, the delivery vehicles are targeted to specific tissues or cells in animals. These compositions and methods are useful in the treatment of conditions where destruction of specific tissues is desirable, such as for treatment of cancers or unwanted vasculature. In some instances, the pore-forming effect can be used as an adjunct to other therapies, for example, by opening the blood-brain barrier, increasing vascular endothelial permeability, or by abetting cell entry by therapeutic or diagnostic agents.

Thus, in one aspect, the invention is directed to a composition of lipid-based delivery vehicles associated with one or more conjugates comprising the formula:

blocking segment−protease target−pore-forming segment,

and wherein a particulate delivery vehicle may be included in the blocking segment.

In some embodiments, said delivery vehicles contain a targeting agent and/or ligand specific for an intended tissue or cell target. The word “comprising” is used in describing the conjugate as it may be necessary to couple the conjugate to a moiety that assures its association with a particulate delivery vehicle. In most cases, such moiety is coupled to or included in the blocking segment, but in some cases this moiety can be coupled to the pore-forming compound or to the linker. The moiety can be of various lengths and descriptions and may include a spacer. If a delivery vehicle is included in the blocking segment, the composition may (or may not) include additional delivery vehicles.

The word “comprises” is also used because, as shown below, it is possible to bracket the pore-forming segment with dual protease targets, thus enhancing the specificity of the construct. Thus, in constructs where there is a protease target on either side of the pore-forming fragment and blocking segments at either end of the molecule, the action of high levels of protease on both protease targets would be required to liberate the activity. The specificity is particularly enhanced if different proteases are required. Thus, this embodiment comprises the generic formula

blocking segment−protease target−pore-forming segment−protease target−blocking segment

where “blocking segment,” “protease target” and “pore-forming segment” are defined as above, and again, an additional moiety for coupling to delivery vehicles, if needed, could also be included somewhere in the molecule.

In another aspect, the invention is directed to a method of effecting cell growth inhibition, necrosis or apoptosis by administering the compositions of the invention in vivo to an animal.

In still another aspect, the invention is directed to a method to prepare the compositions of the invention by admixing the conjugate prodrug with preformed delivery vehicles in a suspension, or by including the conjugate or a portion thereof with the step of formation of the delivery vehicles.

In still another aspect, the invention is directed to methods to personalize treatment of cancer or elimination of other unwanted tissue by assessing the protease content of said cancer or tissue and designing the prodrug so that the protease associated with the conjugate is cleaved by the protease present in high amount in the target.

### BRIEF DESCRIPTION OF THE DRAWINGS

FIGS. 1A-1C show the effectiveness of the blocking segment in reducing cytotoxic and hemolytic activity of melittin coupled through a linker to said blocking segment.

FIGS. 2A-2D show that the conjugate can be cleaved by MMP-9 to restore cytotoxic and hemolytic activity.

FIGS. 3A and 3B show that MMP-9 can cleave both nanoparticle-bound prodrug and free prodrug at the expected site.

FIGS. 4A-4D show the results of tumor treatment with prodrug-coupled nanoparticles. FIGS. 4A and 4B are graphical representations of effect on tumor volume and weight respectively and FIGS. 4C and 4D are ultrasound images.

### MODES OF CARRYING OUT THE INVENTION

The present invention represents an improvement both of the prior art emulsions which contained active pore-forming/cytolytic peptides and prior art conjugates of such peptides with blocking segments coupled through protease-cleavable linkers. In the present invention, the particulate emulsion overcomes substantial problems experienced with using the conjugates alone. Similarly, providing the pore-forming/cytolytic peptide or other compound as a prodrug conjugate also enhances the specificity and effectiveness of this delivery system, and permits personalized treatment.

Because the conjugate is associated with particulate, it is protected from degradation in the blood. Second, because it is associated with a particulate, the administered emulsion is processed through the liver and spleen where the prodrugs which are not delivered to the desired location are degraded. (Particulate delivery of drugs other than peptides does not necessarily have this effect since the liver or spleen may not be able to process them as it does peptides.) Third, because of the lipid-based nature of the particles, the conjugate is delivered directly to the cell membrane. If the particles further contain a targeting agent, this membrane delivery may be made even more specific to a desired tissue.

It should be noted that the particle must just be in the proximity of the tumor in order to effect cytolysis. Thus, if a targeting agent is used, it need not target tumor cells directly, but rather some other marker in the environment or the neovasculature that is usually associated with tumor cells. The delivery vehicles must just be in sufficient concentration in the proximity of tumor cells in order to kill them. Thus, if the constructs are in sufficiently high concentration in and around the tumor matrix where the appropriate protease is released to activate the system and release the pore-forming segment, the segment will diffuse to kill the tumor cells and other tumor helper cells such as macrophage. Thus, any problem with poor penetration of the delivery vehicles to the exact location of the tumor cells is not an impediment, while the specificity is maintained due to the elevated production of proteases appropriate to the protease target associated with the tumor or its environment.

Importantly, the particulate delivery vehicles provide additional methods to control the effective dosage of the pore-forming compound at the intended delivery site, thus providing nuanced timing of delivery of the pore-forming, cytolytic compound to the intended target. This is accomplished in several ways.

First, the presence of a multiplicity of conjugates associated with a delivery vehicle permits a steady supply of conjugate for cleavage at the target site. The number of conjugates associated with a single particle can range in value from one to several thousand depending on the size of the particle and the nature of the coupling. Typical numbers are in this range and could be 10, 20, 50, 100, 1,000, and all integers in between. Of course, not all of the particles in a single emulsion will have the same number of conjugates associated with them, and the numbers here would represent an average over the particles contained in an emulsion. The number of prodrug conjugates that can be associated with an individual delivery vehicle depends, of course, on the nature of the delivery vehicle itself and its dimensions, as well as the mechanism for associating the prodrug with the vehicle.

Second, because the nature of the linkage to the particles can be adjusted, the rate of cleavage can be adjusted as well. The closer the protease target contained in the conjugate is to the surface of the particle, the more slowly the cleavage proceeds, since the steric features will determine the accessibility of the protease target to the protease itself. By inserting spacers into the conjugate, such as polyethylene glycol of various lengths, the protease site can be distanced from the surface of the particle at any desired distance. Third, the protease target, itself, may also be modified so that the rate at which the protease is able to cleave it is changed. Thus, by replacing one or two amino acids in the recognized target, the rate can be altered.

Delivery Vehicles

The delivery vehicles of the invention may be based on any lipid-based microparticles or nanoparticles.

For example, the vehicles may be embodied as various alternative nanoparticulate compositions containing hydrophobic cores and lipid/surfactant coating are described in U.S. Pat. Nos. 6,676,963; 7,255,875 and 7,186,399, all incorporated herein by reference and these descriptions need not be repeated here. The nanoparticles comprise cores of perfluorocarbons that remain liquid in vivo and the cores are coated with lipid/surfactant. These patents also refer to earlier compositions used as contrast agents.

Delivery vehicles may also include other lipid carriers such as liposomes, lipid micelles, lipoprotein micelles, lipid-stabilized emulsions and polymer-lipid hybrid systems. Liposomes can be prepared as described in Liposomes: Rational Design (A. S. Janoff, ed., Marcel Dekker, Inc., N.Y.), or by additional techniques known to those knowledgeable in the art. Liposomes for use in this invention may be prepared to be of “low-cholesterol,” i.e., either “cholesterol free” wherein the liposomes are prepared in the absence of cholesterol, or contain “substantially no cholesterol” that allows for the presence of an amount of cholesterol that is insufficient to significantly alter the phase transition characteristics of the liposome (typically less than 20 mol % cholesterol). Liposomes may also contain therapeutic lipids, which include ether lipids, phosphatidic acid, phosphonates, ceramide and ceramide analogues, sphingosine and sphingosine analogues and serine-containing lipids.

Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Use of cholesterol-free liposomes containing PG or PI to prevent aggregation increases the blood residence time of the carrier.

Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins, et al., Int. J. Pharm. (2000) 200:27-39 (incorporated herein by reference). Lipoprotein micelles can be prepared from natural or artificial lipoproteins including low and high-density lipoproteins and chylomicrons.

Lipid-stabilized emulsions are micelles prepared such that they comprise an oil filled core stabilized by an emulsifying component such as a monolayer or bilayer of lipids. The core may comprise fatty acid esters such as triacylglycerol (corn oil). The monolayer or bilayer may comprise a hydrophilic polymer lipid conjugate such as DSPE-PEG. These delivery vehicles may be prepared by homogenization of the oil in the presence of the polymer lipid conjugate. Agents that are incorporated into lipid-stabilized emulsions are generally poorly water-soluble. Synthetic polymer analogues that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used (Lavasanifar, et al., J. Biomed. Mater. Res. (2000) 52:831-835).

General methods of preparing nanoparticles and microparticles are described by Soppimath, et al. (J. Control Release (2001) 70:1-20) the contents of which are incorporated herein.

Polymer-lipid hybrid systems consist of a polymer nanoparticle surrounded by a lipid monolayer, wherein the lipid monolayer provides a stabilizing interference between the hydrophobic core and the external aqueous environment. Polymers such as polycaprolactone and poly(d,1-lactide) may be used while the lipid monolayer is typically composed of a mixture of lipid. Suitable methods of preparation are well known in the art.

By “associated with” delivery vehicles is meant that the conjugate (or other moieties associated with the delivery vehicles) are bound to the delivery vehicles in such a way that they can be successfully delivered to a target without being removed from the delivery vehicle. The association is typically non-covalent as the conjugates may be provided with moieties, or may inherently include moieties, that are easily non-covalently bound to the delivery vehicle. Since the delivery vehicles of the invention are lipid-based, typically a hydrophobic region or moiety will be present. However, in some cases, the conjugate may be covalently bound to the particles or to one of the components of the particles. In fact, the blocking segment may include a particle covalently bound to the remainder of the conjugate. In the case of liposomes, the conjugates may be encapsulated or associated with the lipid bilayer.

The compositions of the invention are typically administered intravenously so that the liver/spleen system efficiently degrades the conjugate that is not provided to the desired location. Administration of the compositions is thus usually parenteral, although in some indications, oral administration may be employed. Other methods include nebulization and introduction to the airway epithelium

In short, the particulate invention compositions permit effective use of prodrug conjugates of pore-forming, cytolytic compounds, such as peptides, in vivo. The subjects of such in vivo treatment are animals in general—i.e., organisms with cells and tissues that are enveloped by a cell membrane and not protected by cell walls. Such subjects include, for example, mammals, including humans, livestock, companion animals, laboratory model systems such as rodents, rabbits, and guinea pigs, avian subjects such as poultry, and fish.

Personalized Treatment

Of particular importance is the ability of the invention compositions to be designed specifically for treatment of an individual patient or other subject based on the nature of the tissue to be destroyed. Typically, the tissue will be tumor tissue or other undesired tissue and will have associated with it elevated levels of particular proteases that are not found in normal tissue or are found in lesser concentrations. These proteins will differ from tumor to tumor, from individual to individual and over the course of tumor progression. Thus, by obtaining a sample from an individual subject of tumor tissue and determining the protease composition as compared to normal tissue, the protease target can be chosen to match the specificity of the protease(s) present at higher levels.

The determination of the levels of protease that are favorable in the desired target tissue can be conducted by any means known in the art. One might assay, for example, for particular proteases using standard activity assays and comparing tumor tissue to normal tissue. Alternatively, proteomics or genomic studies could be employed. The levels of specific proteases may be ascertained by chromatographic behavior or by interaction with appropriate antibodies or other specific binding partners. Expression levels may also be obtained, at least to a first approximation by determining the transcriptome of the cancer in comparison to normal tissue. While it is understood that there is not a perfect correlation between mRNA levels and the levels of protein produced, at least a first approximation can be obtained using this method and then verifying the results using an appropriate assay for the protein.

This approach differs from typical attempts at personalized medicine where the nature of the pharmaceutically active agent is selected to match the abnormal physiology or metabolic network of an individual's tumor or where antibodies are chosen, for example, based on receptors or other ligands that are associated with tumors—i.e., tumor-associated antigens. Even therapies that are based on the presence or absence of certain enzymes present in the subject, such as enzymes that will cleave irinotecan to its active component SN-38, differ from the present invention in concept.

The present approach also has the advantage that the activity of the bioactive agent is separate from the properties that confer specificity. Thus, in the case of standard pharmaceutical or chemotherapeutic approaches, a small molecule could be used to inhibit an enzyme that is abnormally active in propagating the tumor. In this case, the specificity of the drug and its activity are intertwined. However, in the present case, the activity of the pore-forming segment is generic. The specificity is conferred elsewhere by the nature of the protease target. The role of the protease itself in tumor progression is not significant since it is used only to confer specificity while the antitumor activity of the pore-forming segment is generic to any target cell. This simplifies selection and drug development in that no particular target need be identified for both the specificity and the activity of the drug.

The Conjugates

As noted above, the conjugates that are delivered by the particulate emulsions of the invention comprise the formula

blocking segment−protease target−pore-forming segment,

where the conjugate may (or may not) further contain a moiety to assist it in associating with the selected delivery vehicle, and wherein the particulate delivery vehicle may be included in the blocking segment, so as to sterically block the activity of the pore-forming segment. As noted above, such moieties may be coupled at any location in the conjugate, but are preferably coupled to or included in the blocking segment, such as coupled to the N-terminus of the blocking segment if the blocking segment is a peptide or are included in the blocking segment or are coupled to another accessible part of a moiety that is a non-peptide blocking segment. Such moieties are typical lipophilic moieties such as, for example, phosphatidyl ethanol, etc., or could simply be fatty acid esters, etc. The moiety, especially when included in the blocking segment, may also contain a spacer such as PEG depending on the control of kinetics. The details of these components are set forth below.

Because a number of different chemical entities can serve as blocking segments—i.e., are capable, when included in the conjugate of inactivating the pore-forming segment—a multiplicity of formats for the conjugate may be envisioned.

In one form of the conjugate, the blocking segment may be, for example, a hydrophobic peptide which is sufficiently hydrophobic to serve as a moiety compatible with the delivery vehicles. In this case, the conjugate consists of a blocking segment−protease target−pore-forming segment.

However, if this is not the case and the selected blocking segment is not sufficiently compatible with the delivery vehicle surfaces to effect association with the delivery vehicles, an additional compatibility-conferring moiety may be coupled to the blocking segment or to the pore-forming segment. Preferably it will be coupled to or included in the blocking segment.

In some embodiments, the blocking segment, while not derived, for example, from a pro sequence, is a non-peptide hydrophobic entity such as a phospholipid or cholesterol or a glycolipid. In this case, the conjugate, again, consists of the formula

blocking segment−protease target−pore-forming segment.

In still another embodiment, the blocking moiety may itself be a nanoparticle. In this case, further inclusion of delivery vehicles may then be unnecessary, but the compositions preferably include additional delivery vehicles of the various types set forth above.

When the particulate itself serves as a blocking segment by sterically inhibiting activity of the cytolytic segment, the cytolytic segment is joined via the cleavable linker to a component of the nanoparticle, such as a phospholipid, sphingolipid, ceramide, glycolipid, glycoprotein, synthetic peptide/peptoid or cholesterol derivative. Protease activity would then liberate the cytolytic segment from the particulate, allowing the cytolytic effect. Additionally, an inert spacer, such as polyethylene glycol chains of various lengths, may be incorporated between the particle and protease target in order to control the rate of cleavage of the linker by environmental proteases.

In one embodiment, the nanoparticle is a perfluorocarbon nanoemulsion, the spacer is a polyethylene glycol chain with molecular weight 2000 Da, the protease target is cleavable by MMP-9 (e.g., GPQGIAGQ), and the cytolytic segment is melittin joined to the remainder of the construct at its N-terminus.

In all of the above cases, the blocking segment may include an inert spacer or an inert spacer may be included in the protease target so as to adjust the distance between the protease target effective location from the delivery vehicle. The following embodiments are exemplary, but not limiting. The moiety compatible with delivery vehicle will be designated MCDV.

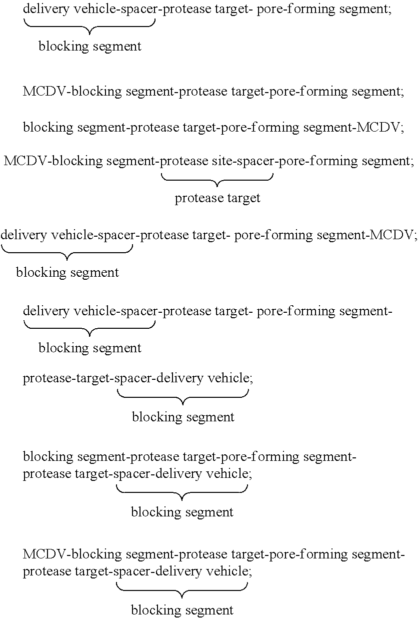

In the last three examples, there is a dual targeting system to improve specificity. Thus, elevated levels, for example, of two different proteases (or indeed the same protease) would be required in order to effect cytolytic activity. This provides enhanced specificity to the constructs.

The various embodiments of the portions of the conjugate are interchangeable and one can mix-and-match the possibilities to obtain alternative embodiments also within the scope of the invention.

Pore-Forming Segment

The pore-forming segment is a compound that is typically a peptide. As used herein, the word “peptide” is not intended to impose an upper limit on the number of amino acids contained. Any peptide/protein which is capable of effecting cell penetration can be used in the methods of the invention. However, for practical reasons, peptides that are relatively short are conveniently used in the methods and compositions of the invention since they are more readily synthesized. Thus, the length of the peptide may be as short as 10-20 amino acids, or the peptide may contain 50 amino acids, 100 amino acids, 200 amino acids and more and all integer numbers in these ranges. The nature of the delivery vehicle can be adjusted to provide a suitable environment for the peptides/proteins used in the invention depending on the specific characteristics thereof. Thus, the nature of the lipids and surfactants used in these vehicles are selected so as to accommodate cationic peptides, anionic peptides, neutral peptides, hydrophobic peptides, hydrophilic peptides, amphipathic peptides, etc.

A pore-forming peptide, which is also cytolytic, can include a number of embodiments well known in the art. Useful peptides include, besides melittin, classic pore-forming peptides such as magainin and alamethicin (Ludtke, S. J., et al., Biochemistry (1996) 35:13723-13728; He, K., et al., Biophys. J. (1996) 70:2659-2666). Pore-forming peptides can also be derived from membrane active proteins, e.g., granulysin or prion proteins (Ramamoorthy, A., et al., Biochim Biophys Acta (2006) 1758:154-163; Andersson, A., et al., Eur. Biophys. J. (2007) DOI 10.1007/s00249-007-0131-9). Other peptides useful in the invention include naturally occurring membrane active peptides such as the defensins (Hughes, A. L., Cell Mol Life Sci (1999) 56:94-103). The D-amino acid analogs of the conventional L forms may be included in synthetic peptides, especially peptides that have all of the L-amino acids replaced by the D-enantiomers. Peptidomimetics that display cell pore-forming, cytolytic properties may be used as well. Thus other peptides useful in the pore-forming segment include “pore-forming peptides” include both natural and synthetic peptides and peptidomimetics.

Magainin, protegrin, LL-37, MSI-78, dermaseptin, cecropin, caerin, ovispirin and alamethicin have been described by Brogden, K. A., Nat Rev Microbiol. (2005) 3:238-250. δ Endotoxin, colicin, aerolysin and α Hemolysin have been described by Gilbert, R. J. C., Cell Mol Life Sci CMLS. (2002) 59:832-844. Amoebapore has been described by Leippe, M, et al., Mol Microbiol. (1994) 14:895-904. Pardaxin has been described by Oren, Z, et al., Eur J Biochem. (1996) 237:303-310. GALA has been described by Li, W., et al., Adv Drug Deliv Rev. (2004) 56:967-985. Nisin and subtilin have been described by Kordel, M., et al., FEBS Lett. (1989) 244:99-102.

One particular class of pore-forming peptides useful in the invention has the general characteristics of melittin in that its members each comprises a hydrophobic region of 10-20 amino acids adjacent to a cationic region of 3-6 amino acids. Melittin itself is formed from a longer precursor in bee venom and mature melittin has the amino acid sequence

(SEQ ID NO: 1)
GlyIleGlyAlaValLeuLysValLeuThrThrGlyLeuPro-
AlaLeuIleSerTrpIleLysArgLysArgGlnGln-NH₂.

Various analogs of melittin has been identified and tested as described in U.S. Pat. No. 5,645,996, for example. Other designs for pore-forming peptides useful in the invention will be familiar to those in the art. In the melittin analogs, the hydrophobic region is preferably 15-20 amino acids long, more preferably 19-21 and the cationic sequence is preferably 3-5 or 4 amino acids long.

Melittin is a water-soluble, cationic, amphipathic, acid alpha-helical peptide. Suchanek, G., et al., PNAS (1978) 75:701-704. It constitutes 40% of the dry weight of the venom of the honey bee Apis mellifera. Although a candidate for cancer chemotherapy in the past, melittin has proved impractical because of its non-specific cellular lytic activity and the rapid degradation of the peptide in blood. Attempts have been made to stabilize melittin by using D-amino acid constituents (Papo, N., et al., Cancer Res. (2006) 66:5371-5378) and melittin has been demonstrated to enhance nuclear access of non-viral gene delivery vectors (Ogris, M., et al., J. Biol. Chem. (2001) 276:47550-47555 and Boeckle, S., et al., J. Control Release (2006) 112:240-248). The ultimate effect of melittin is to cause the formation of pores in a cell membrane, and possibly membranes of internal cell organelles, so as to damage the cell and lead to cell death.

In another embodiment a peptide from the Bcl-2-family proteins is employed as the pore-forming peptide in the conjugate based on activating or inhibitory activity (Danial, N. N., et al., Cell (2004) 116:205-219). After penetrating to the cellular interior the peptides cause activation or inhibition of the endogenous Bcl-2-family or associated proteins in the cells (Walensky, L. D., et al., Mol Cell (2006) 24:199-210). Thus, the cellular machinery of apoptosis can be to a variety of therapeutic goals.

The pore-forming peptides in the conjugates may also have an anti-infective role, since they are generally toxic to bacteria, fungi and viruses. These peptides are considered host-defense peptides so that the circulating peptides will have a bactericidal, fungicidal, or antiviral activity.

The toxicity of such peptides is affected by a number of factors, including the charge status, bending modulus, compressibility, and other biophysical properties of the membranes as well as environmental factors such as temperature and pH. The presence or absence of certain moieties (including those other than any targeted epitope) on the cell surface may also affect toxicity.

For clarity, it is noted that typically the “pore-forming peptides” included in the conjugates of the invention are also cytolytic. In some instances, therefore, “pore-forming,” “cytolytic” and “pore-forming/cytolytic” are used interchangeably in describing these active portions of the prodrug. Generally speaking, these peptides are also cytotoxic. This description is true of pore-forming segments that are non-peptide as well.

Synthetic membrane lytic non-peptide compounds have also been described (Gokel, G. W., et al., Bioorganic & Medicinal Chemistry (2004) 12:1291-1304). Additional antimicrobial peptides, many of which are pore-forming toxins, are listed in the APD2 Database at aps.unmc.edu/AP/main php.

The Blocking Segment

The blocking segment is typically but not necessarily itself a peptide. One embodiment is the blocking segment derived from pro-melittin itself, which has the prodomain APEPEPAPEPEAEADAEA DPEA (SEQ ID NO:2). Truncated forms of this prodomain may also be used. Also available, as disclosed in the paper by Holle, cited above, is the latency associated peptide (LAP) that forms a protective segment with respect to TGF-β, Yu, et al., Int. J. Oncol. (2009) 35:829-835. Other peptide sequences that are useful as blocking segments include propeptide fragments representing the prodomain of other peptides cytokines, hormones, and other biologically active peptides. In addition, polyglutamate sequences of various lengths, may be used as well as any large protein such as avidin. Also useful as a blocking segment is the particle itself covalently fused to the toxin thus serve to sterically block pore formation and reduce cytotoxicity. That is, the conjugate may use the particulate itself as a blocking mechanism in which case the blocking segment includes the particulate delivery vehicles, covalently bound to the remainder of the conjugate.

The blocking segment may also include, or may itself be, a moiety that is compatible with the delivery vehicle. Such moieties are hydrophobic, and can include phospholipids, lipid moieties such as cholesterol, or may be sphingolipids, ceramides, glycolipids or other hydrophobic moieties. Peptidomimetics may also be included or may be the blocking segment. The blocking segment must merely confer, by its presence in the conjugate, the inability of the pore-forming segment to perform its function. The blocking segment may also include a neutral spacer such as polyethylene glycol (PEG) to effect the rate of hydrolysis or other cleavage of the prodrug.

Thus, in some embodiments, the blocking segment may include or may be a phospholipid such as phosphatidylglycerol, phosphatidylinositol, phosphatidyl ethanolamine, etc., or may be or may include cholesterol or other lipids, or lipoproteins. In any case, “blocking segment” results in inactivation of the pore-forming segment and may also serve the dual function of the capacity to couple non-covalently to the delivery vehicle or may concomitantly comprise the delivery vehicle itself.

Protease Target

The protease cleavage site included in the linker between the blocking segment and the pore-forming peptide is an amino acid sequence that represents a target for a desired protease. Thus, among suitable sequences are the target sequence for MMP-9, which is GPQGIAGQ (SEQ ID NO:3), other MMP's, such as MMP-2 the target sequence for fibroblast activation protein (FAP), and the target sequences for legumain, and plasmin.

Depending on the specific tumor cells or tumor in a subject, one or more of these or other proteases may be elevated, and the protease target segment will be designed to be a suitable substrate for this particular protease. In addition, tumors may be associated with a reducing environment so that disulfide linkages could be used or may generate acid so that a pH-dependent linkage for hydrolysis could be employed. Typically, however, the targeted linker will be a specific amino acid sequence susceptible to protease cleavage.

Thus, a variety of conjugates suitable for use in the invention can be constructed by varying the three components of the conjugate.

Methods of Use

For the conjugates, advantage can be taken simply of the cytotoxic effects of the pore-forming peptides. In treating tumors, for example, it is desirable to effect growth inhibition or cell death specifically on the malignant cells or to exert similar effects on the neovasculature associated with such tumors. Other conditions associated with unwanted neovasculature, such as ocular conditions including age related macular degradation are treatable using the compositions of the invention. Other conditions that can be successfully treated using the compositions of the invention are conditions of the cardiovascular system, and in some cases, conditions of the brain. As stated above, the particulates of the invention protect the prodrugs from degradation, in some cases reduce the toxic effect on bystanders due to processing by the liver, are helpful in associating the conjugate with the cell membrane, and permit fine-tuning of the rate of pore-formation and cytolyses.

Additional Components

In one embodiment, the delivery vehicles, such as liposomes, micelles, or other lipid-based microparticles or nanoparticles may also contain additional therapeutic agents or diagnostic agents for which cell entry is desired. In this case, the delivery vehicles comprising such additional components are generally vehicles that are not integral to the blocking segment. Thus, the delivery vehicles may also contain small molecule drugs, oligonucleotides such as antisense nucleic acids or gene silencing RNA, nucleic acid vectors, radioisotopes, fluorescent compounds, and the like.

Multiple types of drugs can be included, including drugs which effect positive outcomes, such as angiogenic agents, for example, VEGF, or antiproliferatives, such as paclitaxel. Generally speaking, these delivery mechanisms are employed for any pharmaceutical in the pharmacopeia and more than one type of drug may be delivered, for example, by including a multiplicity of drugs in association with a single particulate delivery system. Alternatively, a composition for administration may be composed of particles bearing different drugs or diagnostic agents targeted to the same tissue, not targeted, or targeted to different tissues with or without combination with non-targeted particles. Compositions of the invention can be mixed and matched in this manner. Alternatively, various compositions either containing single types of particles or mixtures of different types may be administered in sequence.

In one form of this embodiment, one or more compounds or additional peptides to be delivered to a desired target cell or tissue may also be included in the delivery vehicles. Liposomes and nanoparticles, including targeted nanoparticles, are known as vehicles for such administration, but the effectiveness of delivery is further enhanced by including the conjugate containing pore-forming compound. Thus, a multiplicity of drugs or combination of drugs and diagnostic agents may be included in the particle delivered to the cells or tissue. Compositions containing a multiplicity of particles of various types also are included within the scope of the invention. Thus, penetration of barriers to entry of the therapeutic or diagnostic compound is enhanced across the cell membrane or even across the blood-brain barrier.

The conjugate may be associated with moieties to be delivered into the cell by non-covalent association. For example, DNA or RNA which are negatively charged, can be associated with a positively charged amino acids included in a pore-forming peptide. They may also be associated with, for example, lipid/surfactant layers that are themselves positively charged. Plasmids, including expression vectors, can thus be transfected into cells by association with the conjugates of the invention.

In some embodiments, the compositions of the invention also contain, in the delivery vehicles, a targeting ligand. The targeting ligand is specific for a target cell or tissue.

As used herein, a ligand “specific” for a target cell or tissue means simply that the ligand binds sufficiently more tightly to the target than to non-targeted cells or tissues to exert its effect substantially only on the target. Typically, this binding is through an epitope exhibited on the surface of the target cell or tissue. Typical targeting agents include antibodies, aptamers, peptides, peptidomimetics and the like and are also described in the above mentioned Lanza patent, as are means for coupling such targeting agents to the nanoparticles. Typically such techniques involve coupling the targeting ligand, usually covalently, to a moiety, which can be absorbed into the delivery vehicles. Thus, the targeting ligand is often covalently coupled to a component of the delivery vehicles or a lipid/surfactant layer, which is itself non-covalently included in this layer.

Preparation

The conjugate is simply absorbed into the delivery vehicle, or in some cases, includes the delivery vehicle. In one method of preparation of the invention, the suspension of delivery vehicles, optionally containing the targeting agent, is mixed with the appropriate amount of conjugate and incubated for a sufficient length of time to effect absorption. This preparation method has the advantage of permitting the emulsion to be sterilized prior to the addition of conjugate, avoiding conditions that would degrade the conjugate itself.

However, if the particle serves as a blocking segment it is covalently bound to the protease target and is an integral part of the blocking segment.

Alternatively, the coupling of the conjugate to the delivery vehicles may occur simultaneously with the formation of the emulsion containing delivery vehicles. In this scenario, the conjugate and the lipids used to form the delivery vehicles may be included in the same hydrophobic composition which is then emulsified or otherwise treated to form the delivery vehicles in a one-step process. If the delivery vehicles are liposomes, for example, the hydrophobic composition which comprises the liposome components and the conjugates or conjugates which include the components of the liposomes may be subjected to sonication or other known techniques for forming liposomes. If the delivery vehicles are microparticles or nanoparticles containing liquid hydrocarbon or fluorocarbon cores, the core materials can be emulsified with the hydrophobic composition containing the conjugates and additional lipid/surfactant to obtain the already coupled delivery vehicles.

Thus the cytolytic prodrug conjugate may be either directly incorporated into the nanoparticle during synthesis or joined to the nanoparticle in a post-formulation step using well-established chemical conjugation or adsorption procedures.

In another alternative, the delivery vehicles containing just the blocking segment or blocking segment coupled only to the protease target or a portion thereof may be assembled into the nanoparticles when the nanoparticles or other delivery vehicles are formed, and then subsequently coupled to the remainder of the conjugate using standard coupling techniques, including, particularly favorably, Click chemistry. Thus, for example, one member of the conjugate can be supplied with an alkyne and the other member with an azide moiety resulting in the formation of a triazole in the standard Click chemistry reaction.

Various methods to obtain the ultimate compositions of the invention will be known to the practitioner.

In sum, the invention compositions offer, for the first time, the opportunity to effect cell barrier crossing in a selective manner without hemolysis of red blood cells or destruction of tissues whose existence is desirable. In addition, and importantly, the invention provides for personalized treatment of individuals based on the composition of that individual's tumor.

As used herein, “a”, “an” and the like indicate one or more than one unless otherwise indicated or obvious from the context.

The following examples are offered to illustrate but not to limit the invention.

### Example 1

### Preparation of Perfluorocarbon Nanoparticles

A. Perfluorocarbon nanoparticles were synthesized as described by Winter, P. M., et al., Arterioscler. Thromb. Vasc. Biol. (2006) 26:2103-2109. Briefly, a lipid surfactant co-mixture of egg lecithin (90 mol %) and dipalmitoyl-phosphatidylglycerol (DPPG; 10 mol %) (Avanti Polar Lipids, Piscataway, N.J.) was dissolved in chloroform, evaporated under reduced pressure, dried in a 50° C. vacuum oven and dispersed into water by sonication. The suspension was combined with either perfluoro-octylbromide (PFOB), or perfluoro-15-crown ether (CE) (Gateway Specialty Chemicals, St. Peters, Mo.), and distilled deionized water and continuously processed at 20,000 lbf/in² for 4 min with an S110 Microfluidics emulsifier (Microfluidics, Newton, Mass.).

B. If targeted nanoparticles are used, one embodiment employs αᵥβ₃-integrin. These nanoparticles are made by incorporating 0.1 mole % peptidomimetic vitronectin antagonist conjugated to polyethylene glycol (PEG)₂₀₀₀-phosphatidylethanolamine (Avanti Polar Lipids, Piscataway, N.J.) replacing equimolar quantities of lecithin.

The αᵥβ₃-integrin targeting ligand linked to phosphatidyl ethanolamine has the formula:

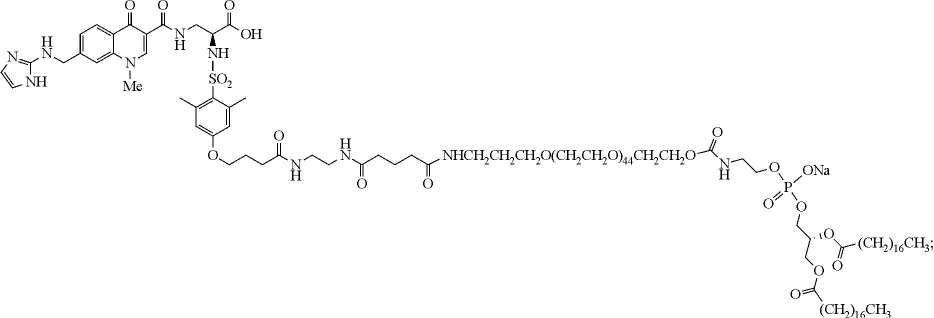

### Example 2

### Preparation of Melittin Conjugate

A conjugate of the formula:

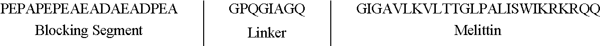

The conjugate was compared to melittin and to melittin coupled only to linker in order to determine toxicity to endothelial cells and macrophages, as well as hemolysis. For assessment of cytotoxicity, 2F2B endothelial cells (CRL-2168; American Type Culture Collection, Manassas, Va.) or RAW 264.7 macrophages (TIB-71; American Type Culture Collection, Manassas, Va.) were plated in a 96-well plate at a concentration of 15,000 cells/well and allowed to attach overnight. The peptide of interest was then added and incubated with the cells for 3 hours at 37° C. Following this incubation, the agent of interest was removed, cells were washed with phosphate-buffered saline, and cell viability was determined by XTT assay (Biotium, Hayward, Calif.). This assay measures the ability of cellular oxidoreductases to reduce the XTT reagent, resulting in a colored product. Briefly, cells were incubated with the activated XTT reagent for 3 hours at 37° C. with shaking at 200 rpm. The absorbance of each well at 450 nm was then measured using a Bio-Rad Model 550 plate reader (Bio-Rad Laboratories, Hercules, Calif.) with reference wavelength 630 nm.

For hemolysis measurements, citrated whole blood was collected from New Zealand white rabbits and separated by centrifugation at 2000 rpm for 10 minutes. Red blood cells were washed five times with Dulbecco's phosphate buffered saline and stored at 4° C. until use. In each assay, the peptide of interest was added to 1×10⁷ red blood cells in PBS and incubated at 37° C. for one hour. Afterwards, intact red blood cells were pelleted by centrifugation at 2000 rpm for 5 minutes and absorbance of the supernatant at 450 nm was measured using a Bio-Rad Model 550 plate reader (Bio-Rad Laboratories, Hercules, Calif.) with reference wavelength 630 nm To determine percent lysis, absorbance readings were normalized to lysis with 0.1% Triton™ X-100.

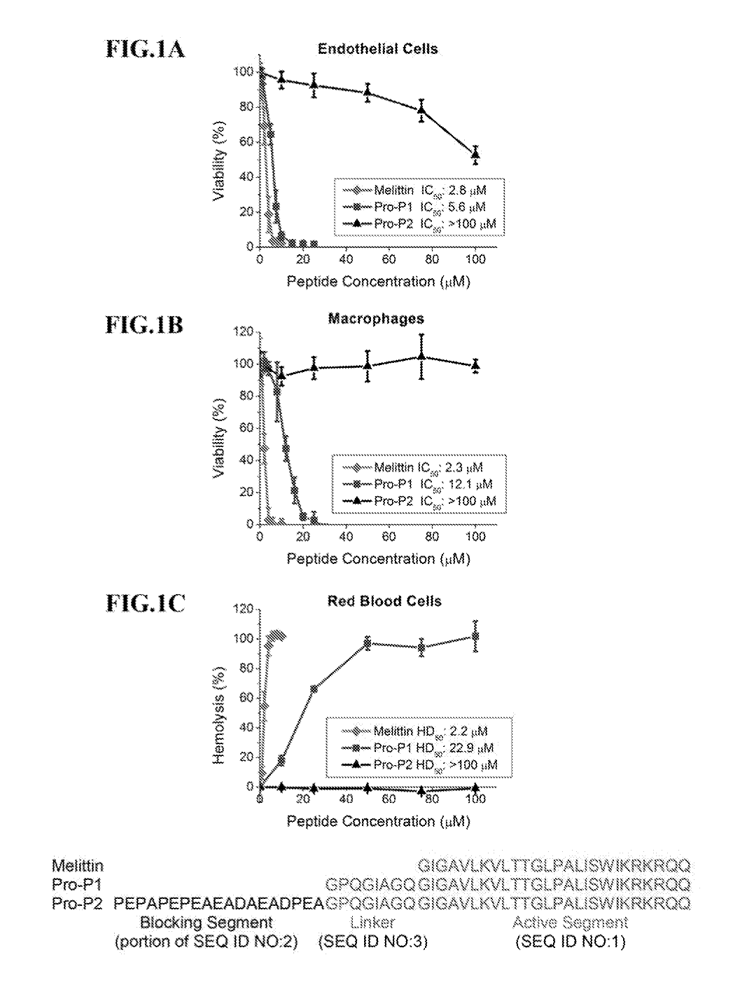

Results of these experiments are shown in FIGS. 1A-1C. For all cell types, addition of the linker alone (Pro-P1) decreased the cytolytic activity of melittin while addition of the linker and blocking segment (Pro-P2) reduced melittin cytotoxicity and hemolytic activity by at least two orders of magnitude.

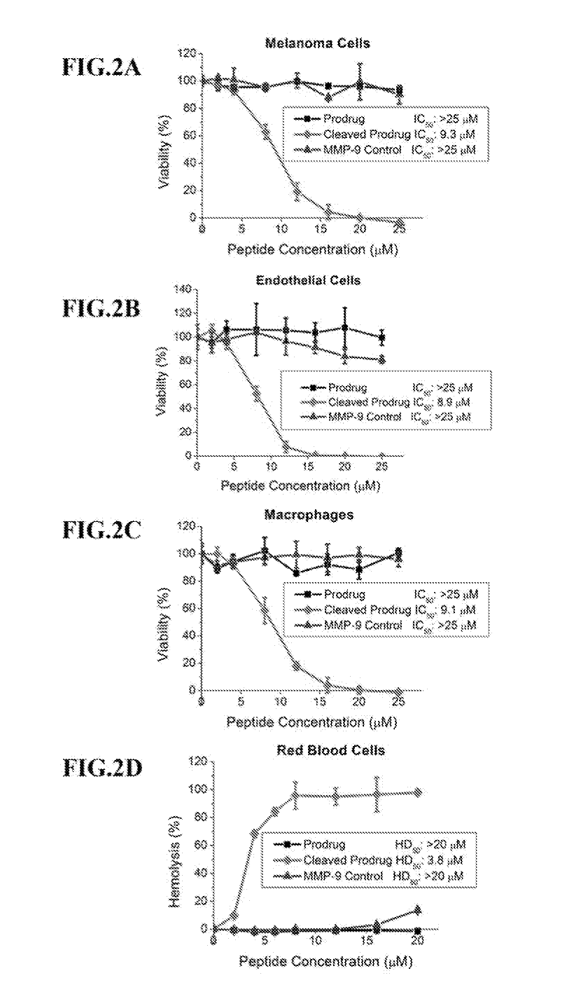

To assess the impact of proteolytic cleavage on conjugate cytotoxicity and hemolytic activity, we repeated these experiments following cleavage of the conjugate by matrix metalloproteinase-9 (MMP-9). Recombinant MMP-9 (R&D Systems, Minneapolis, Minn.) was activated by incubation with 1 mM 4-aminophenylmercuric acetate (APMA; Sigma-Aldrich, St. Louis, Mo.) at 37° C. for 2 hours. Free APMA was removed by dialysis against TCNBZ buffer (150 mM NaCl, 50 mM Tris, 10 mM CaCl₂, 10 μM ZnCl₂, 0.05% (w/v) Brij-35, pH 7.75) for 2 hours using a Slide-A-Lyzer MINI Dialysis Device, 2K MWCO (Thermo Scientific, Waltham, Mass.) with a dialysate change at 1 hour. The conjugate was cleaved by incubation at a concentration of 200 μM with 20 nM MMP-9 in TCNBZ buffer for 3 hours at 37° C. As shown in FIGS. 2A-2D, cleavage of the conjugate by MMP-9 restored its toxicity to B16F10 melanoma cells (CRL-6475; American Type Culture Collection, Manassas, Va.), 2F2B endothelial cells, and RAW 264.7 macrophages, as well as its hemolytic activity.

### Example 3

### Incorporation of Conjugate onto Nanoparticles

Conjugate-loaded nanoparticles were formulated by mixing known amounts of conjugate to non-targeted perfluorocarbon nanoparticles prepared in Example 1A and compared to melittin loading. Both melittin and the conjugate were produced by solid-phase peptide synthesis (melittin: GenScript, Piscataway, N.J.; conjugate: American Peptide Company, Sunnyvale, Calif.). Perfluorocarbon nanoparticles were incubated with 1 mM melittin or conjugate in phosphate-buffered saline at 4° C. for 1 hour. Nanoparticles were then isolated by centrifugation at 5,000 rpm for 15 minutes. The supernatant was removed and nanoparticles were washed three times with phosphate-buffered saline to remove free peptide. The loading efficiency of melittin and the conjugate were comparable, resulting in approximately 16,000 peptides per nanoparticle in each case.

### Example 4

### Characterization of Conjugate-Loaded Nanoparticles

### A. Size Distribution and Zeta Potential

The size and zeta potential (0 of conjugate-loaded nanoparticles of Example 3 were measured on a ZetaPlus zeta potential analyzer (Brookhaven Instruments, Holtsville, N.Y.). Size distribution was determined by photon correlation spectroscopy while zeta potential was determined by electrophoretic mobility analysis. Data were acquired in the phase-analysis light scattering (PALS) mode following solution equilibration at 25° C. The Smoluchowski approximation was employed to calculate from the measured nanoparticle electrophoretic mobility (μ):

μ=∈·ζ·(1.5)/η

where ∈ and η are the dielectric constant and the absolute viscosity of the medium, respectively.

Control nanoparticles exhibited a size of 292.7 nm and a zeta potential of −64.6 mV. Loading with either melittin or the conjugate did not substantially alter the size of these nanoparticles (melittin-loaded nanoparticles: 287.5 nm; conjugate-loaded nanoparticles: 290.6 nm). In contrast, loading with melittin caused a significant positive shift in nanoparticle zeta potential (26.4 mV) due to the cationic nature of this peptide. Loading with the conjugate did not significantly change nanoparticle zeta potential (−65.2 mV), as expected due to the relatively neutral overall charge of this peptide.

### B. Nanoparticle-Bound Peptide Characterization

Characterization of nanoparticle-bound peptides prepared in Example 3 was accomplished through disruption of nanoparticle structure by addition of isopropanol and subsequent analysis by high-performance liquid chromatography (HPLC). Reversed-phase HPLC of peptide mixtures was performed using a Waters system (Waters In-Line Degasser, Waters 626 Pump, Waters 717 plus Autosampler, Waters 600S Controller, Waters 2487 Absorbance Detector) and a Vydac 218TP54 (C18) column The mobile phase consisted of a mixture of 0.1% trifluoroacetic acid (TFA) in water (solvent A) and 0.1% TFA in acetonitrile (solvent B). Composition of the mobile phase was varied from 60% buffer A/40% buffer B to 40% buffer A/60% buffer B over the course of 20 minutes. A flow rate of 1.2 mL/min and injection volume of 20 μL were used. Eluting peptides were detected by absorbance at 215 nm.

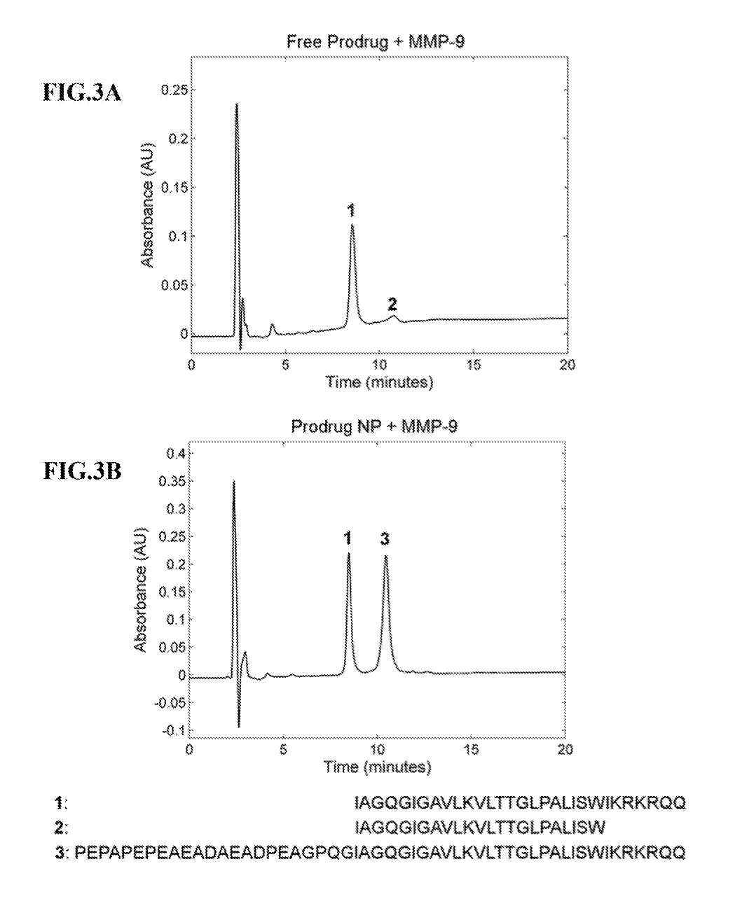

In order to assess cleavage of the free and nanoparticle-bound conjugate, free conjugate and conjugate-loaded nanoparticles were incubated with recombinant MMP-9 as described in Example 2 and characterized by HPLC analysis. Eluting peptide fractions were collected and analyzed by mass spectrometry to determine peptide identity. FIG. 3A-B shows representative chromatograms of free and nanoparticle-bound conjugate following cleavage by MMP-9, as well as the sequences of peptides corresponding to each chromatogram peak. Both free and nanoparticle-bound conjugate are successfully cleaved to yield the expected product (peak 1). However, cleavage of nanoparticle-bound conjugate proceeds more slowly as demonstrated by the presence of residual uncleaved conjugate (peak 3) following exposure to MMP-9.

### Example 5

### Interaction of Conjugate-Loaded Nanoparticles with Cells

The effects of conjugate-loaded nanoparticles on B16F10 melanoma cell viability in the presence and absence of MMP-9 were studied by XTT assay as described in Example 2. The conjugate-loaded nanoparticles of Example 3 were used. Neither the free conjugate nor conjugate-loaded nanoparticles were toxic to cells in the absence of MMP-9. Addition of MMP-9 to culture media caused both the free conjugate and conjugate-loaded nanoparticles to become substantially cytotoxic to melanoma cells. The % of viable cells was reduced to 20% by the nanoparticle prodrug treated with MMP-9 and essentially to 0% by prodrug/MMP-9 combination.

### Example 6

### Effect of Conjugate-Loaded Nanoparticles in Tumor Models: B16 Melanoma

In this model, one million B16F10 melanoma cells were implanted in the right inguinal region of C57BL/6 mice on day 0. Three groups of mice with five mice in each group were employed. The control group was treated with saline. A second group was treated with blank nanoparticles which were prepared as described in Example 1A but which were not loaded with conjugate. The third group was treated with conjugate-loaded nanoparticles prepared as described in Example 3.

The animals were dosed on days 5, 7, 9 and 12 by tail vein injection. The dose in group 3 was 1 mg/kg of conjugate while the dose in group 2 was an equal volume of emulsion that did not contain conjugate.

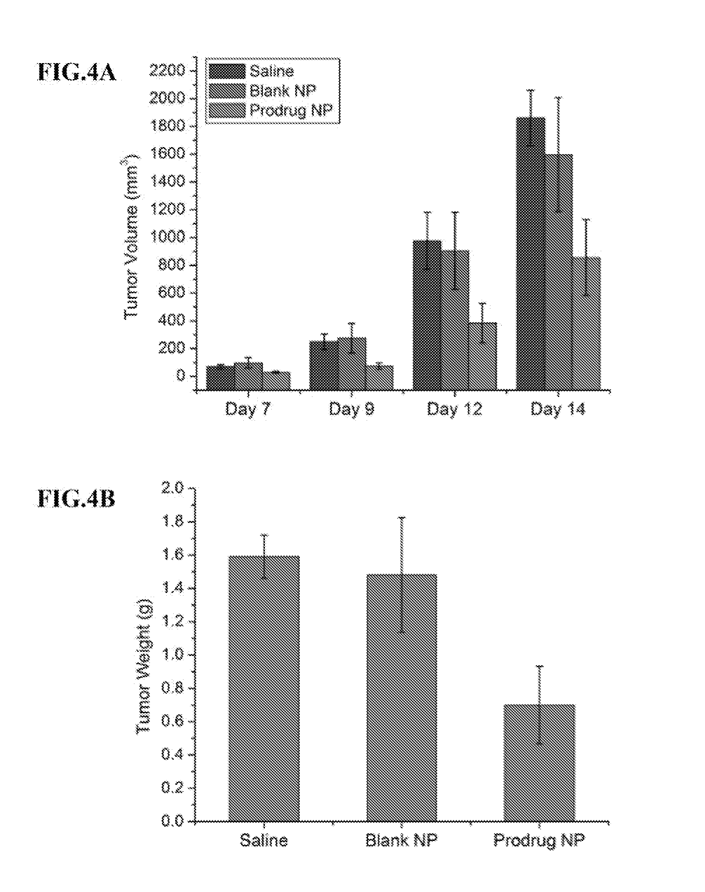

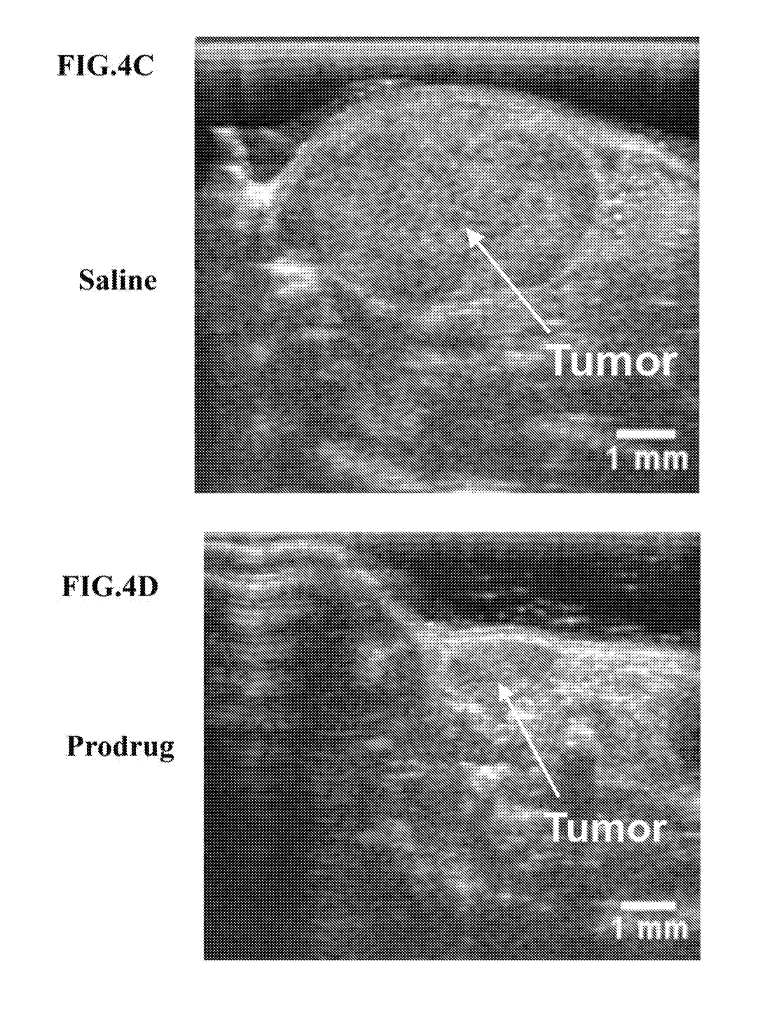

The mice were imaged with ultrasound on days 7, 9, 12 and 14, and were sacrificed on day 14. Tumor volumes, end tumor weights, and sample ultrasound images are shown in FIGS. 4A-4D.

As shown in FIG. 4A, the end volumes in cubic millimeters (mm³) were as follows:

Saline: 1,861 (±200);

Blank nanoparticles: 1,596 (±410); and

Conjugate nanoparticles: 856 (±273).

Similarly, the end tumor weights in grams as shown in FIG. 5B were as follows:

Saline: 1.59 (±0.13);

Blank nanoparticles: 1.48 (±0.34);

Conjugate nanoparticles: 0.70 (±0.23).

As shown, a significant reduction in tumor growth rate (p=0.043) was obtained with conjugate-loaded nanoparticles.

1. A method to prepare a medicament suitable for administration to an individual cancer subject, which method comprises determining the protease content of a sample of cancerous tissue from said subject in comparison to the protease content of normal tissue and preparing a composition comprising particulate delivery vehicles which are associated with a conjugate comprising the formula (1):
blocking segment−protease target−pore-forming segment (1)
or of formula (1a)
blocking segment−protease target−pore-forming segment−protease target−blocking segment (1a)
wherein said protease target is selected to match a protease identified in said cancer tissue whose content is elevated in comparison to normal tissue, and
wherein in said conjugate the blocking segment deactivates the pore-forming segment, and
wherein said particulate delivery vehicle is optionally included in the blocking segment.

2. A composition comprising particulate delivery vehicles which are associated with a conjugate comprising the formula (1):
blocking segment−protease target−pore-forming segment (1)
or of formula (1a)
blocking segment−protease target−pore-forming segment−protease target−blocking segment (1a)
wherein in said conjugate the blocking segment deactivates the pore-forming segment, and
and wherein said particulate delivery vehicle is optionally included in the blocking segment.

3. The composition of claim 2 wherein the conjugate further comprises a hydrophobic moiety for association with said delivery vehicles.

4. The composition of any of claim 2 wherein the pore-forming segment is a peptide.

5. The composition of claim 4 wherein the peptide comprises a hydrophobic amino acid sequence of 10-30 amino acids adjacent to a cationic amino acid sequence of 3-6 amino acids.

6. The composition of claim 5, wherein the pore-forming peptide is melittin.

7. The composition of claim 2, wherein the blocking segment comprises a peptide, a phospholipid, a lipid and/or a delivery vehicle and optionally a spacer.

8. The method or composition of claim 7 wherein the blocking segment comprises a delivery vehicle.

9. The composition of any of claim 7 wherein the blocking segment comprises a spacer.

10. The composition of claim 2, wherein the protease target is a target for an MMP, FAP, legumain or plasmin.

11. The composition of claim 2, wherein the delivery vehicles are liposomes, micelles or nanoparticles which nanoparticles comprise a liquid hydrophobic core coated with a lipid/surfactant layer.

12. The composition of claim 2, wherein said delivery vehicles further include a therapeutic or diagnostic agent, and/or wherein the delivery vehicles further contain a targeting ligand specific for a target tissue or cell.

13. A method to treat a condition in a subject benefited by destruction or inhibition of the growth of a target cell or tissue which method comprises administering to said subject the composition of claim 2.

14. A method to treat a condition in a subject benefited by delivery of a therapeutic agent to a target cell or tissue which method comprises administering to said subject the composition of claim 12 wherein the delivery vehicles further include said therapeutic agent, or a method to diagnose a condition in a subject which method comprises administering to said subject the composition of claim 12 wherein the delivery vehicles further include said diagnostic agent.

15. A method to prepare the composition of claim 2, which method comprises
(a) incubating particulate delivery vehicles with said conjugate comprising the formula (1)
blocking segment−protease target−pore-forming segment (1)
or comprising the formula (1a)
blocking segment−protease target−pore-forming segment−protease target−blocking segment (1a)
wherein said delivery vehicles are not included in the blocking segment(s),
whereby said conjugate is associated with said delivery vehicles; or
(b) forming an emulsion containing said delivery vehicles in the presence of said conjugate; or
(c) coupling delivery vehicles to which a portion of the conjugate is bound to the remaining portion of the conjugate under conditions wherein the conjugate is formed.

16. The method of claim 2 wherein the conjugate further comprises a moiety for association with said delivery vehicles.

17. The method of claim 1 wherein the pore-forming segment is a peptide which comprises a hydrophobic amino acid sequence of 10-30 amino acids adjacent to a cationic amino acid sequence of 3-6 amino acids.

18. The method of claim 1 wherein the blocking segment comprises a peptide, a phospholipid, a lipid and/or a delivery vehicle and optionally a spacer.

19. The method of claim 1 wherein the delivery vehicles are liposomes, micelles or nanoparticles which nanoparticles comprise a liquid hydrophobic core coated with a lipid/surfactant layer.

20. The method of claim 1 wherein said delivery vehicles further include a therapeutic or diagnostic agent, and/or wherein the delivery vehicles further contain a targeting ligand specific for a target tissue or cell.


SEQUENCE LISTING (3 sequences)

SEQ ID NO: 1 - protein, 26 aa, Apis mellifera
GIGAVLKVLTTGLPALISWIKRKRQQ

SEQ ID NO: 2 - protein, 22 aa, Apis mellifera
APEPEPAPEPEAEADAEADPEA

SEQ ID NO: 3 - protein, 8 aa, Apis mellifera
GPQGIAGQ